(19) United States
(12) Patent Application Publication
(10) Pub. No.: US 2021/0012881 A1
(43) Pub. Date: Jan. 14, 2021

(54) SYSTEMS, METHODS AND APPARATUS FOR TREATMENT PROTOCOLS

(71) Applicant: Soteria, LLC, Brookfield, CT (US)

(72) Inventors: Patrick Lydon Queenan, Brookfield, CT (US); David Mancusi, Bethel, CT (US)

(21) Appl. No.: 16/917,086

(22) Filed: Jun. 30, 2020

Related U.S. Application Data
(60) Provisional application No. 62/869,506, filed on Jul. 1, 2019.

(51) Int. Cl. G16H 20/70; G16H 40/67; G16H 10/60; G16H 50/30; G16H 50/70; G06F 16/23; G06N 20/00; G09B 19/00; A61B 5/00

(52) U.S. Cl. CPC G16H 20/70; G16H 40/67; G16H 10/60; G16H 50/30; G16H 50/70; G06F 16/2379; G16H 50/20; G09B 19/00; A61B 5/0002; A61B 5/4035; A61B 5/742; A61B 5/4836; A61B 5/4848; G06N 20/00

(57) ABSTRACT

System, method and apparatus for enabling caregivers to predict when people need supports, make better clinical decisions in the moment, provide more supports remotely and/or directly through technology independent of caregivers, and learn the optimal intervention for each individual in order to prevent crises and enhance outcomes. This may be done through capturing autonomic nervous system, biometric, environmental, and clinical data and applying machine learning to determine predictive modeling equations on a per-person basis with prompts for the person, activation of remote monitoring systems, modifications to the environment, or alerts to caregivers the moment that a predictive variable exceeds its individualized statistical control limit.

### CLAIM TO PRIORITY

This application claims priority to U.S. Provisional Application 62/869,506 filed Jul. 1, 2019, which is hereby incorporated by reference in its entirety herein.

### FIELD OF THE DISCLOSURE

The present disclosure relates to a system, method and apparatus to record, predict, and prevent certain behaviors. Additionally, the disclosure describes enhanced care delivery through sensed data, machine learning, and automated prompts.

### BACKGROUND

Today, society is faced with a variety of mental health issues and there are serious concerns how to best provide care for mental health patients.

### BRIEF SUMMARY

Embodiments described herein are directed to systems, methods and apparatus to allow for caregivers to: predict when people (patients) need supports; make better clinical decisions in the moment; provide more supports remotely and/or directly through technology independent of caregivers; and learn the optimal intervention for each individual in order to prevent crises and achieved optimal outcomes. As used herein, patients, people and individuals refers to people and patients under, or being considered for, a treatment.

### BRIEF DESCRIPTION OF THE DRAWINGS

The accompanying drawings, which are incorporated in and constitute a part of this specification, illustrate embodiments of the present disclosure and, together with a general description given above, and the detailed description given below, serve to explain the principles of the present disclosure.

FIG. 1 shows an example of inputs and outputs according to one embodiment of the disclosure.

FIG. 2 shows an example of an administrative portal and a behavior recording application according to another embodiment of the disclosure.

FIG. 3 shows an example of prompts according to yet another embodiment of the disclosure.

FIG. 4 shows an embodiment of control charts and push notifications according to an embodiment of the disclosure.

FIG. 5 shows a representation of an autonomic nervous system visualization according to an embodiment of the disclosure.

FIG. 6 shows a representation of heart rate variability push notifications according to an embodiment of the disclosure.

FIGS. 7A, 7B and 7C show examples of protocols according to an embodiment of the disclosure.

FIG. 8 shows an example of how the components act together to avert a crisis situation according to an embodiment of the disclosure.

FIG. 9 shows a series of steps to avert a crisis situation for a patient according to an embodiment of the disclosure.

It should be understood that the appended drawings are not necessarily to scale, presenting a somewhat simplified representation of various features illustrative of the basic principles of the disclosure. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The present disclosure will now be described more fully hereinafter with reference to the accompanying drawings, in which preferred embodiments are shown. This disclosure may, however, be embodied in many different forms and should not be construed as limited to the embodiments set forth herein. Rather, these embodiments are provided so that this disclosure will be thorough and complete, and will fully convey the scope to those skilled in the art.

Those of ordinary skill in the art realize that the following descriptions of the embodiments of the present disclosure are illustrative and are not intended to be limiting in any way. Other embodiments will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the disclosure. Accordingly, the following embodiments are set forth without any loss of generality to, and without imposing limitations upon, the claims.

In this detailed description, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present disclosure.

Furthermore, in this detailed description, a person skilled in the art should note that quantitative qualifying terms such as “generally,” “substantially,” “mostly,” “approximately” and other terms are used, in general, to mean that the referred to object, characteristic, or quality constitutes a majority of the subject of the reference. The meaning of any of these terms is dependent upon the context within which it is used, and the meaning may be expressly modified.

The present disclosure addresses shortcomings of the current practices in managing patient treatment protocols. The disclosure however is not limited in its applicability to these systems. Embodiments described herein relate to an improved monitoring system to address a national staffing shortage, and a very high rate of staff turnover in the mental health care industry, which compromises providers' ability to support people with disabilities and suffering from mental health issues and problems.

Typically, Applied Behavior Analysis has been limited to operate from a univariate perspective, while acknowledging that most behaviors are multiply determined. Thus, it is typical to attempt to isolate one antecedent at a time and measure or rate the impact on a target behavior, and to trial one intervention at a time after hypothesizing the function of that behavior.

Currently, Applied Behavior Analysis often operates under the assumption that one intervention, when successful, will generally be successful in all conditions. However, behavior is situation-specific. Current technology typically involves utilizing numerous mediating and moderating variables simultaneously, test interventions, record responses, and synthesizing the variables, interventions and responses to reach a determination of the most effective interventions or treatments in a variety of situations.

The present disclosure relates to a system, method and apparatus to record, predict, and prevent certain behaviors. Additionally, the disclosure describes enhanced care delivery through data parameters, machine learning, Internet of Things (IoT) and automated prompts; and provides visualizations to simplify complex treatment protocols. The visualizations of treatment options allow minimally trained health care providers, or even laypersons to provide care to a patient.

People, and patients, with complex medical, psychiatric, and behavioral issues are at risk for being cared for in a suboptimal manner, resulting in greater acuity and expense to an already strained system. Subjective reporting is the norm for treatment of specific disorders. For example, when undergoing anxiety treatment with exposure therapy, people are asked to rate on a “Subjective Units of Distress Scale” how intense their anxiety is. The issue is that it is not known, nor readily discernable, whether this is a high anxiety condition relative to this person's baseline when this data is reported subjectively. Thus, a subjective scale is less than desirable since the scale depends on a qualitative parameter. This qualitative parameter is based largely on an opinion of the patient regarding their condition. Objective measures would make treatment more effective, and efficient.

Furthermore, people and patients often become dependent on caregivers to deliver prompts to them, and therefore remain at higher levels of care since the patients do not develop the ability to do things independently. This dependence upon a caregiver results in a diminished quality of life since the patient may be confined to an in-patient treatment facility when the patient, with proper support could be treated on an out-patient basis or a family member could administer the necessary therapy, or treatment to avoid a crisis situation with the patient.

The present disclosure relates to, inter alia:

addressing the national staffing shortage in direct care and reduce overtime costs by reducing the number of caregivers required if more patients are able to live independently with “just-in-time” remote supports;

reducing costs to the provider(s) via reductions in injuries, worker's compensation and property damage if high acuity behaviors are prevented;

reducing costs to the federal government via reductions in the utilization of emergency departments and hospitals, thereby reducing Medicaid and Medicare costs by preventing illnesses and behavioral crises, as well as reduce costs to the surrounding towns via a reduction in utilization of police and EMT services;

reducing costs to the state governments via increased numbers of patients capable of moving from long-term services and supports to supported independent living, shortened lengths of treatment in young adults services;

developing a replicable and portable system so that the gains achieved can be realized by other providers.

One embodiment of this disclosure relates to using individualized analysis for data on a second-to-second time basis to determine when readings are a concern that should be addressed, rather than reliance on population health parameters.

The individualized analysis utilizes data for that particular patient or person to treat the patient or person. Thus, a priori information gathered about the patient is used to determine how to most effectively treat the patient. This approach relates baseline standards and particular idiosyncratic behavior and/or parameters to the particular patient.

Typically in care settings utilize universal parameters that indicate when a condition, or state, is abnormal, but the universal parameters use aggregate data to determine when a condition, or state, is unusual for the population.

The method and model described herein tracks an individual's data over a prolonged period of time and applies a statistical process control chart logic to identify defects to that person's own data so that it is determined what percentage of the time the particular patient should be within certain statistical control limits, and when it is determined that an indication or notification is generated when a parameter for the patient exceeds those limits.

For example, with heart rate, one person may have a resting heart rate of 53 beats per minute and another person may also have a resting heart rate of 53 beats per minute.

A universal parameter would indicate that action needs to be taken when a person's heart rate exceeds 100 beats per minute.

For one person a heart rate of 100 beats per minute may be normal, however because that person's standard deviation is 25 and thus that person's heart beats per minute is still within 2 standard deviations for that person, and this would yield a false positive for them.

However, for another person, there may have been a missed optimal time to intervene because that person has a standard deviation of only 12 points and thus they exceeded their third standard deviation at a heart rate of 89 beats per minute.

Using the parameters that are given on a population level there would still have been something important missing between 89 beats per minute and 100 beats per minute, and have yielded a false negative for that patient. There are likely many false positives and many false negatives when using data on a population or aggregate level.

Embodiments disclosed herein therefore reduce false positives and false negatives by using individualized data collected for a particular patient and repeatedly analyzed over time. The continuous analysis of a specific individual's data provides an enhanced profile for that particular individual.

Another embodiment of this disclosure is directed to application of applied behavior analysis in real-time on a multivariate level. Based on the disclosure herein, it is now possible, on a moment-to-moment basis (seconds as well as milliseconds), to substantially simultaneously investigate multiple sources of environmental and biometric data and the ability to predict problematic behavior.

Analyzing data from multiple sources on a scale of seconds or milliseconds increases the accuracy of prediction of problematic behavior and simultaneously test the efficacy of various interventions by using a library of prompts, and measuring whether the problem behavior occurs or is prevented. The machine learning described herein both learns when problems, or undesired situations, are likely to occur based on multiple inputs and identify which interventions are going to work within those multiply determined events.

Particular embodiments described herein allow caregivers to: predict when patients need supports; make better clinical decisions in the moment; provide more supports remotely and/or directly through technology which can be independent of caregivers; and learn the optimal intervention for each individual in order to prevent crises and thereby enhance outcomes and provide optimal treatment for the patient.

The optimal outcome is achieved through capturing autonomic nervous system (ANS) data, biometric data, environmental data, clinical data as well as any other type of data that is relevant to the patient. Applying machine learning determines predictive modeling equations on a per-patient basis with prompts for the patient, activation of remote monitoring systems, modifications to the environment, or alerts to caregivers the moment that a predictive variable exceeds its individualized statistical control limit.

Additionally, by learning a person's individual variability in different measures of their autonomic nervous system, it is possible to begin to measure a patient's internal state. B. F. Skinner declared that this internal state is “the black box” that was unmeasurable, and needed to be disregarded, with all measurements being concerned instead with observable events.

According to the present disclosure, however, a person's autonomic nervous system (ANS) activity can now be measured; thus opening the “black box” to show a person's own variability, and providing insight when the patient is in an agitated state relative to their own baseline, and thus when intervention is warranted.

The systems and methods described herein recognize that training of caregivers to identify functions of behavior and select appropriate interventions is time consuming and inefficient. Utilization of the systems and methods described herein permit less-well trained personnel and laypeople to provide treatment to patients because the activation of intervention procedures is clear and provides a step-wise approach to defuse a possible crisis situation for the patient.

One embodiment described herein describes machine learning, thus providing caregivers with function-based interventions to trial without the prerequisite training and associated expenses. This is particularly important given the lack of staff, the limited financial resources, and the turnover of staff that is the norm in the mental health care industry.

This disclosure describes providing notifications to a person or patient supported by the technology, as well as to the caregiver(s) when biometric data suggests that there is a concern so that action can be taken. This may prevent medical issues, such as aspirational crises, and predictable psychiatric issues, such as the dramatic sleep changes that occur with the onset of a manic episode of bipolar disorder.

As described herein, this disclosure describes how to eliminate the use of subjective rating scales, which can confound interventions since they are neither reliable nor valid.

Embodiments described herein disclose an application (app) that replaces these subjective measures with objective measures of a person's autonomic nervous system, which will accelerate the application of evidence-based interventions.

The apparatus and system are used to provide a display of patient data that is related to a particular patient.

Embodiments describe systems, methods and apparatus to predict when people need supports, make better clinical decisions in the moment, provide more supports remotely and/or directly through technology independent of caregivers, and learn the optimal intervention for each individual in order to prevent crises and enhance outcomes. This is accomplished by acquiring, obtaining or capturing autonomic nervous system (ANS) data, biometric data, environmental data, clinical data, medical history data, prescription history, allergy information, seizure history information as well as any other pertinent data and applying machine learning to determine predictive modeling equations on a per-person basis with prompts for the person, activation of remote monitoring systems, modifications to the environment, and/or alerts to caregivers the moment that a predictive variable exceeds its individualized statistical control limit.

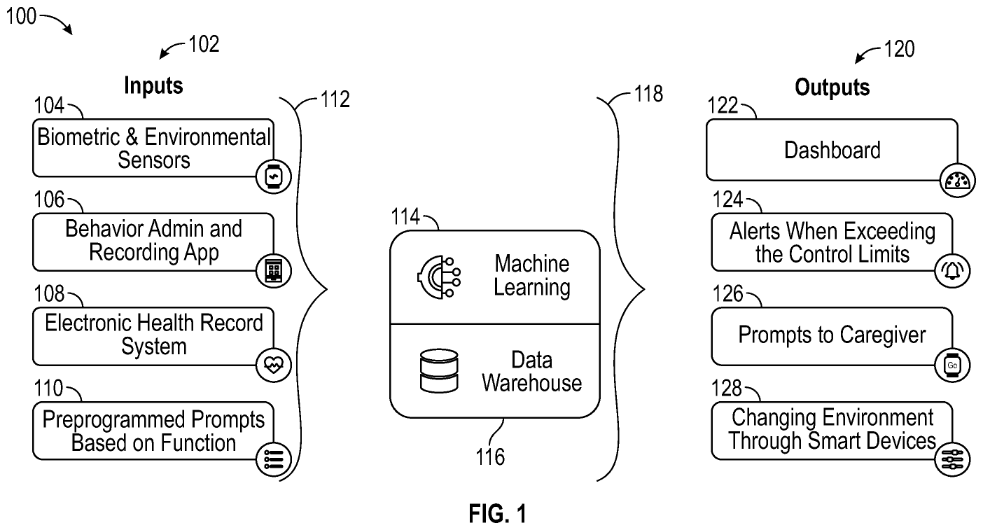

FIG. 1 shows an example 100 according to one embodiment of the disclosure. As shown in FIG. 1, a series of inputs 102, which include biometric and environmental sensors 104, behavior administration and recording application (app) 106, an electronic health record system 108, preprogrammed prompts based on function 110 and other suitable inputs. The inputs 102 may be combined as shown by 112 and are provided to machine learning module 114 and/or data warehouse module 116.

Output from the machine learning module 114 and data warehouse 116, as shown by 118, are shown as outputs 120. The outputs 120 may include dashboard 122, alerts when exceeding the control limits 124, prompts to a caregiver 126 and/or changing environments through one or more smart devices 128. Any combination or permutation of outputs may be generated by the machine learning module 114 and/or data warehouse module 116.

The outputs from the machine learning module 114 and data warehouse module 116 are provided as outputs 120 to an output device, such as a remote memory, such as an electronic storage medium, user interface, such as a graphical user interface, or a touch screen, via interface 118, which may be a bus, wired or wireless connection. The inputs 102 include: biometric sensor data and environmental sensor data; behavior administration and recording app; electronic health record system; and pre-programmed prompts based on function. The inputs are transmitted to machine learning facility 114 and/or data warehouse 116 via interface 112, which may be a wireless, wired or bus or any suitable connection mechanism. The outputs 120 include: dashboard; alerts when exceeding the control limits; prompts to caregiver; and changing environment through smart devices, such as cell phones and other suitable processing devices.

The sensors, such as biometric and environmental sensors 104 may acquire and transmit data via a secure and encrypted data connection. The connection could be between any suitable number of Internet connected devices, terminals, processors or other machines that can transmit and/or receive data via a network.

The biometric sensors are configured to collect, store and transmit biometric data, such as heart rate, pulse rate, body temperature, respiration rate.

Environmental sensors are configured to sense, collect, store and transmit ambient environmental conditions, such as noise, temperature, precipitation, sunlight, and other conditions of an environment of a person, or patient.

Behavior administration and recording app 106 records the frequency, intensity, time of day, duration and antecedents of behavior. This app may be an application, set of instructions, program code, or other computer-executable language that is configured to record, store and transmit types of behavior related to a person, or patient.

Electronic health record system 108 is configured to record diagnostic information treatments administered. This recorded information accumulates what treatments and/or conditions a patient has had in the past or is currently being administered. This recorded information may be transmitted to a data warehouse 116 and/or machine learning facility 114.

The preprogrammed prompts are used to gather additional information regarding the experience of the person, or patient. These may include questions such as last time patient ate, drank, consumed alcohol, took medication, or other information that facilitates diagnosis.

The collected or accessed or acquired data from inputs 102, which includes sensors and apps and records and prompts 104, 106, 108 and 110, respectively, are transmitted to machine learning facility 114 and/or data warehouse 116.

The machine learning module, facility, or set of instructions 114, which may also include associated program code may be integrated with, co-located with, or otherwise in communication with a data warehouse module 116, which may be a management module. For example, the machine learning module 114 may execute on the same host computing device as the data warehouse module 116 and may communicate with the data warehouse module 116 using an API, a function call, a shared library, a configuration file, a hardware bus or other command interface, or using another local channel.

In certain embodiments, the machine learning module 114 may be accessed and/or executed using an extension language, mark-up language, HTML, or interface of the data warehouse module 116 as an extension, plug-in, add-on or the like for the machine learning module 114.

In another embodiment, the machine learning module 114 may be in communication with the data warehouse module 116 via a data network, such as a local area network (LAN), a wide area network (WAN) such as the Internet, as a cloud service, a wireless network, a wired network, or other suitable network.

The machine learning module 114 may comprise computer executable code installed on a computing system for extending, modifying, and/or configuring the data warehouse module 116 with machine learning, predictive functionality.

Additionally, the machine learning module 114 may comprise a dedicated hardware device or appliance or facility in communication with the data warehouse module 116 or facility over the data network, over a communications bus, or other bi-directional wired or wireless communication technique.

The machine learning module 114 is configured to interface with the data warehouse module 116 to extend functionality of the data warehouse module 116 using machine learning. The machine learning module 114, in one embodiment, uses data of the data warehouse module 116 as machine learning inputs, to generate learned functions and/or machine learning ensembles, to provide predictive machine learning results, or the like based on data of the data warehouse module 116.

In one embodiment, the machine learning module 114 may receive data from a user, such as a human user, as configuration data, as a machine learning input, or the like.

Data warehouse 116 is one or more data bases that is configured to access data from many different sources within a defined set of data bases, or other data warehouses, which are controlled by a single entity for reporting and analysis. The data accessed from the data warehouse 116 is used to generate outputs 120. The data warehouse 116 has sufficient processing and electronic storage capacity to produce the outputs 120, via interconnection path 118, which is a wired or wireless connection, such as the Internet, WAN, LAN or other suitable transmission mechanism.

The outputs 120, receive the output from machine learning facility 114 and data warehouse 116 via interconnection 118, which may be wired, wireless, or other suitable interconnection mechanism to provide a transmission path. The outputs 120 may be provided to a user interface of a user device, such as a smart phone, P.C., tablet, or other device having a display. The device may be associated with a patient, or person, or one or more caregivers.

The outputs 120 include a dashboard 122 that is displayed on a user device, such as the screen of a smart phone, tablet, PC or other user interface. The user device can be the person's user device and/or a caregiver's user device.

Alerts 124 are another output that are displayed on a user interface. The alerts may indicate a status or condition of the person, or patient.

Prompts to a caregiver 126 are indicators provided to one or more caregivers regarding the status of a person, or patient. A caregiver may be a friend, bystander, nurse, doctor, or any individual or group of people that assist the patient or person.

Changing environment through smart devices 128 includes an instruction or indicator, displayed on a user interface, such as a smart phone, tablet, PC, or other device with a display, to have the patient or person relocate to another venue or place, such as going outdoors, going into another room indoors, avoiding a crowd, leaving a noisy area, such as a rifle range, music concert, movie theater, playing soft music that is soothing to he person, or patient or other change of stimulus to the person, or patient.

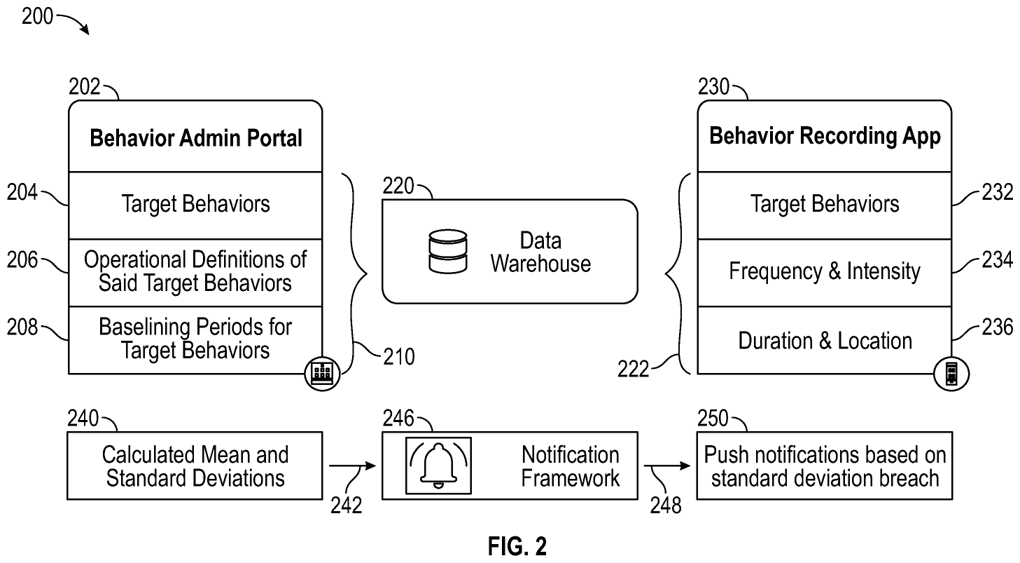

FIG. 2 shows an example 200 according to another embodiment of the disclosure. Specifically, FIG. 2 shows a behavior administration portal 202, a data warehouse 220 and a behavior recording application (app) 230.

The behavior administration portal 202 includes one or more processors and/or one or more electronic memories to store: target behaviors 204; operational definitions of the target behaviors 206; and baseline periods for target behaviors 208. These behavior parameters may be transmitted to data warehouse 220 via interface 210. The data warehouse 220 is similar to data warehouse 116.

The various behaviors parameters 204, 206 and 208 may be combined, as shown by interface 210 and provided to data warehouse 220.

Data warehouse 220 may be located remotely from the behavior administrative portal 202. The data warehouse 220 provides output to behavior recording application 230 via interface 222. Interface 22 is a wired, or wireless or Internet connection or other suitable transmission path between data warehouse 220 and behavior recording application 230.

A behavior app, or recording process 230 is stored on a tangible, electronic storage, such as a non-transitory computer-readable storage medium, such as ROM, PROM, EEPROM thumb drive or any suitable electronic storage medium. Behavior app 230 is an application, which may be a series of programmed steps to perform a specific task or function, when executed.

The behavior app, or recording process 230 measures the occurrence of problematic and pro-social target behaviors, 232 the frequency, intensity 234, duration, location and latency 236 of those behaviors. This information may be accessed from data warehouse 220, or stored in a data warehouse 220, as shown by interface 222.

The behavior recording app 230 includes electronic memories, or storage areas to access and/or store: target behaviors 232; frequency of and intensity of target behaviors 234; and duration of target behaviors and location of target behaviors 236.

Also shown as part of FIG. 2 is a flowchart of operations that are executed by one or more processors and/or one or more electronic memories, which includes, a module that calculates mean and standard deviation quantities 240, a notification framework module 246 and a push notification based on standard deviation breach module 250.

Some embodiments of the present disclosure may be described as a system, method or computer program product. Accordingly, embodiments of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable storage media, such as a non-transitory computer readable storage medium, having computer readable program code embodied thereon.

Many of the functional units described herein have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more physical or logical blocks of computer instructions, which may, for instance, be organized as an object, procedure, or function. Nevertheless, the execution of code of an identified module need not be physically located together, but may comprise disparate instructions stored in different locations which, when joined logically, or operationally, together, comprise the module and achieve the stated purpose for the module.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure.

The operational data may be collected as a single data set, or may be distributed over different locations including over different storage devices, and may exist, at least partially, merely as electronic signals on a system or network. The system or network may include non-transitory computer readable media. Where a module or portions of a module are implemented in software, the software portions are stored on one or more computer readable storage media, which may be a non-transitory media.

Any combination of one or more computer readable storage media may be utilized. A computer readable storage medium may be, for example, but not limited to, an electronic, PROM, ROM EEPROM, magnetic, optical, electromagnetic, infrared, or semiconductor system, apparatus, or device, or any suitable combination of the foregoing, including non-transitory computer readable media.

More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), a Blu-ray Disc, an optical storage device, a magnetic tape, a Bernoulli drive, a magnetic disk, a magnetic storage device, a punch card, integrated circuits, other digital processing apparatus memory devices, or any suitable combination of the foregoing, but would not include propagating signals.

In the context of this disclosure, a computer readable storage medium may be any tangible medium that can contain, or store a program for use by or in connection with an instruction execution system, apparatus, or device.

Program code for carrying out operations for aspects of the present disclosure may be generated by any combination of one or more programming language types, including, but not limited to any of the following: machine languages, scripted languages, interpretive languages, compiled languages, concurrent languages, list-based languages, object oriented languages, procedural languages, reflective languages, visual languages, or other language types.

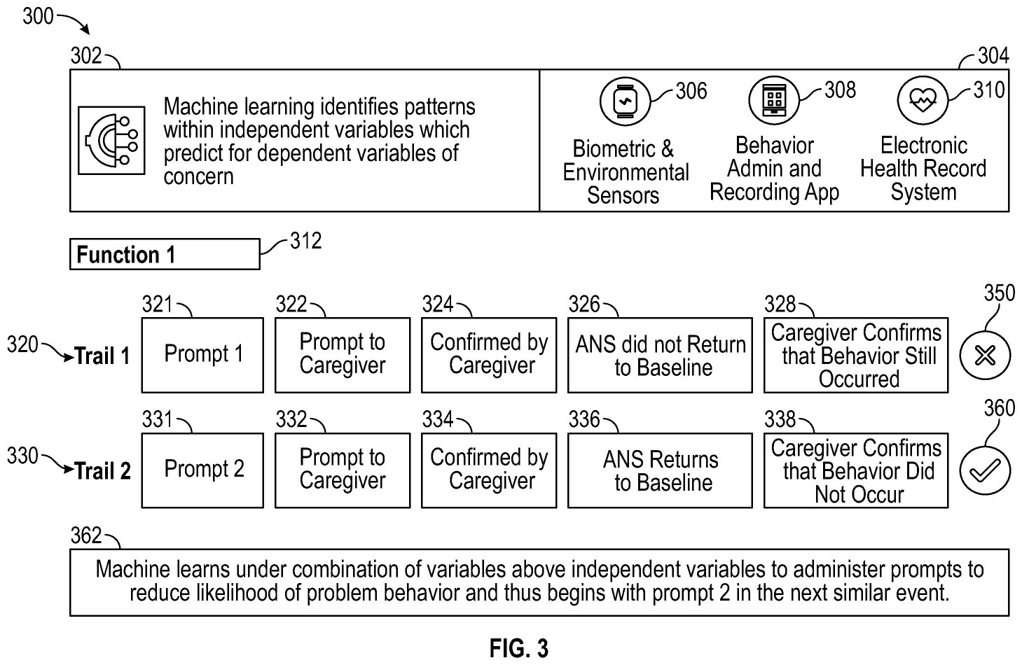

FIG. 3 shows yet another example 300 according to yet another embodiment of the disclosure. FIG. 3 shows trial 1 320 and trial 2 330. Module 302 shows that machine learning identifies patterns within independent variables that predict for dependent variables of concern. Sensor module 304, which is a sensor array, includes biometric and environmental sensors 306, behavior administration and recording application (apps) 308 and electronic health record system 310.

Specifically, sensors 306 provide sensed data related to biometric and environmental sensors of patients and the surrounding area. The biometric sensors sense parameters such as heartbeat, body temperature, pulse blood pressure and other functions. The environmental sensors sense parameters such as ambient temperature, UV level, decibel level vibration, motion and proximity.

Behavior administration and recording app 308 records the frequency, intensity, time of day, duration and antecedents of behavior.

Electronic health record system 310 is configured to record diagnostic information treatments administered.

A functional block 312 includes trial one 320 and trial two 330.

Trial one 320 includes prompt one 321, which includes a prompt to a caregiver 322, confirmation by caregiver 324 an indication that ANS did not return to baseline 326. A caregiver confirms that behavior still occurred 328. A negative indication 350 is generated based on the responses 322, 324, 326 and 328 to prompt 1 321 of trial 1 320.

Trial two 330 includes prompt two 331, which includes a prompt to a caregiver 332, confirmation by caregiver 334 an indication that ANS returned to baseline 336. A caregiver confirms that behavior did not occur 338. A positive indication 360 is generated based on the responses 332, 334, 336 and 338 to prompt 2 331 of trial 2 330.

As shown in FIG. 3, the process utilizes machine learning, as may be performed by machine learning module 114, as shown in FIG. 2, by using a combination of variables to mitigate the likelihood of undesired or problem behavior and the next iteration will begin with prompt two 331 in the subsequent similar event 362. Thus, the positive outcome, indicated as element 360 is utilized in one or more subsequent prompts.

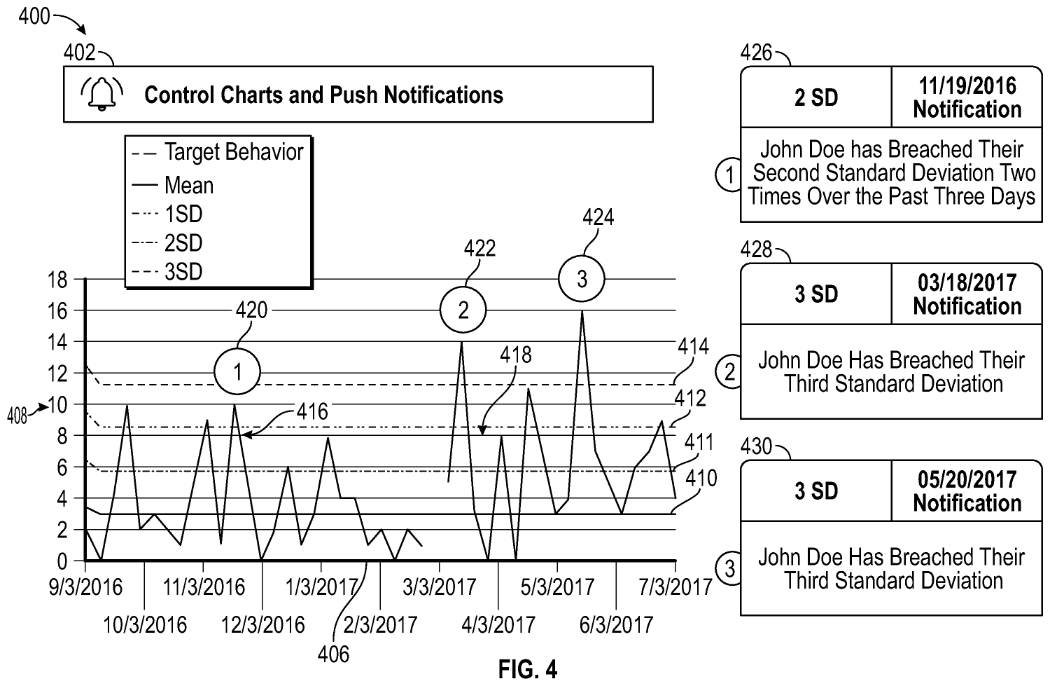

FIG. 4 shows an illustration, using a graph 400, of control charts and push notifications according to an embodiment of the disclosure.

Graph 400 shows time, in months, plotted on x-axis 406 and number of episodes in integers on y-axis 408. Line 416 and line 418 are shown. Points 420, 422 and 424 are depicted on graph 400. Demarcation lines 411, 412 and 414 indicate a standard deviation of a target behavior. Line 410 indicates the average. Line 411 indicates one standard deviation, line 412 indicates a second standard deviation and line 414 indicates a third deviation. Description 426 of point 1 420 indicates that on Nov. 19, 2016, “John Doe has breached his second standard deviation two times over the past three days.” Description 428 of point 2 422 indicates that on Mar. 18, 2017, “John Doe has breached his third standard deviation.” Description 430 of point 3 424 indicates that on May 20, 2017, “John Doe has breached his third standard deviation.”

Line 416 shows a pattern of behavior within control limits. Line 418 shows a pattern of behavior that exceeds control limits.

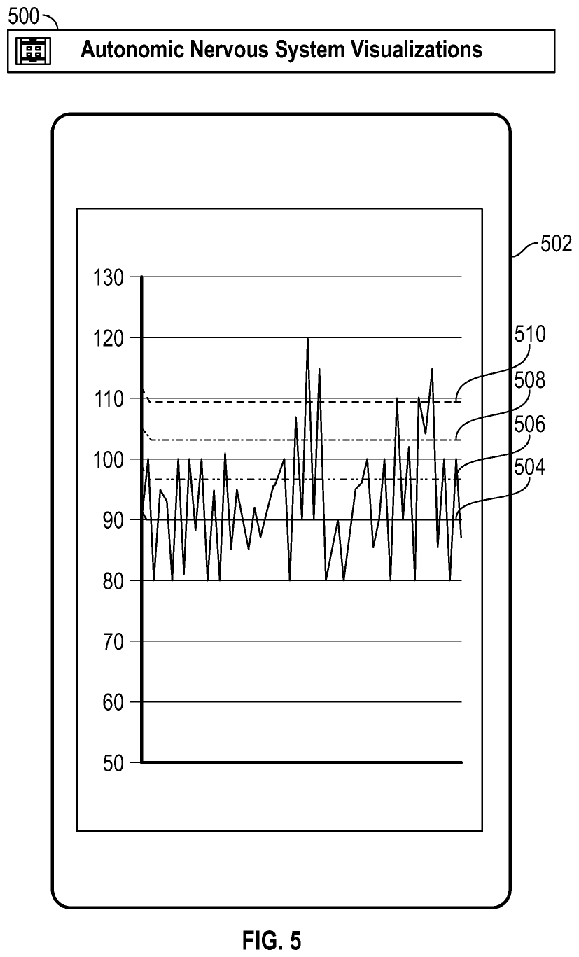

FIG. 5 shows a representation 500 of an autonomic nervous system (ANS) visualization according to an embodiment of the disclosure. FIG. 5 shows a device 502 with a screen. The screen of device 502 displays data 504 to a user. The data displayed 504 includes the data of FIG. 4, as well as other data. The data displayed 504 includes nervous system visualization information. This nervous system visualization information is a graphical representation of patient specific data, outcomes, symptoms, environment, data points relative to the patient's a priori parameters and other indicia of a patient's emotional, mental and/or physical state. The data displayed 504 can indicate the average, first standard deviation, second standard deviation, third standard deviation as well as other health indicators, parameter representations and other relevant information pushed from an interconnected database. The database may utilize a bi-directional communication link, Internet, LAN, WAN, RF or any suitable communication path or conduit.

Screen of device 502 is configured to receive user input, from a user. The screen has menus, and touch screen portions that permit a user to input data that is used at the device 502 and/or transmitted to another processing location.

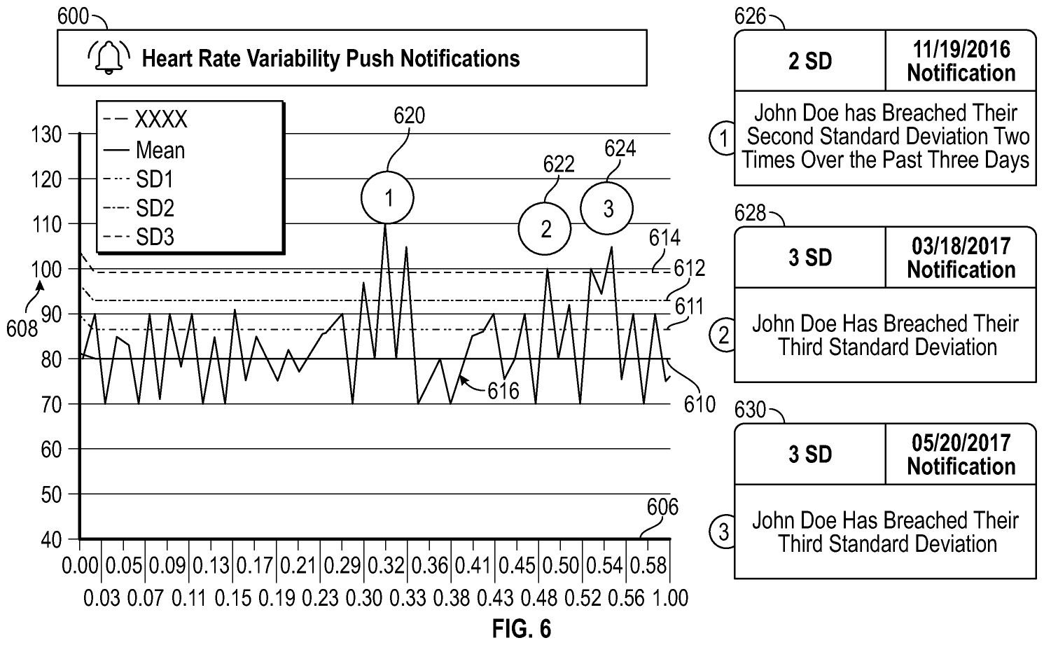

FIG. 6 shows a representation 600 of heart rate variability push notifications according to an embodiment of the disclosure. The points (1 (626), 2 (628) and 3 (630)) correspond to notifications.

Graph 600 shows time, in months, plotted on x-axis 606 and number of episodes in integers on y-axis 608 of heart rate variability push notifications. Line 616 is shown. Points 620, 622, and 624 are depicted on graph 600. Demarcation lines 610, 611 612 and 614 indicate an average, one standard deviation of a target behavior, two standard deviations of a target behavior, and three standard deviations of a target behavior, respectively. Description 626 of point 1 620 indicates that on Nov. 19, 2016, “John Doe has breached his second standard deviation two times over the past three days.” Description 628 of point 2 indicates that on Mar. 18, 2017, “John Doe has breached his third standard deviation.” Description 630 of point 624 indicates that on May 20, 2017, “John Doe has breached his third standard deviation.”

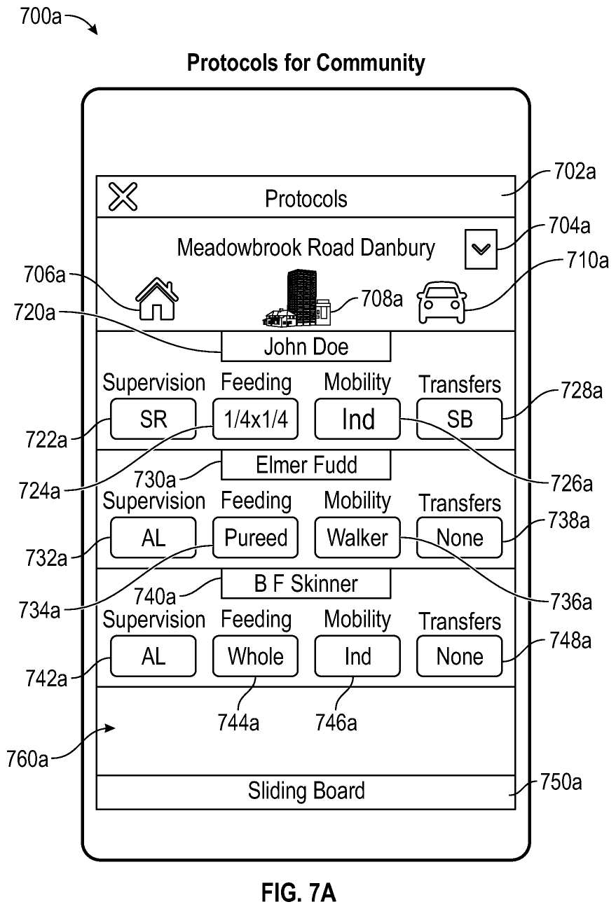

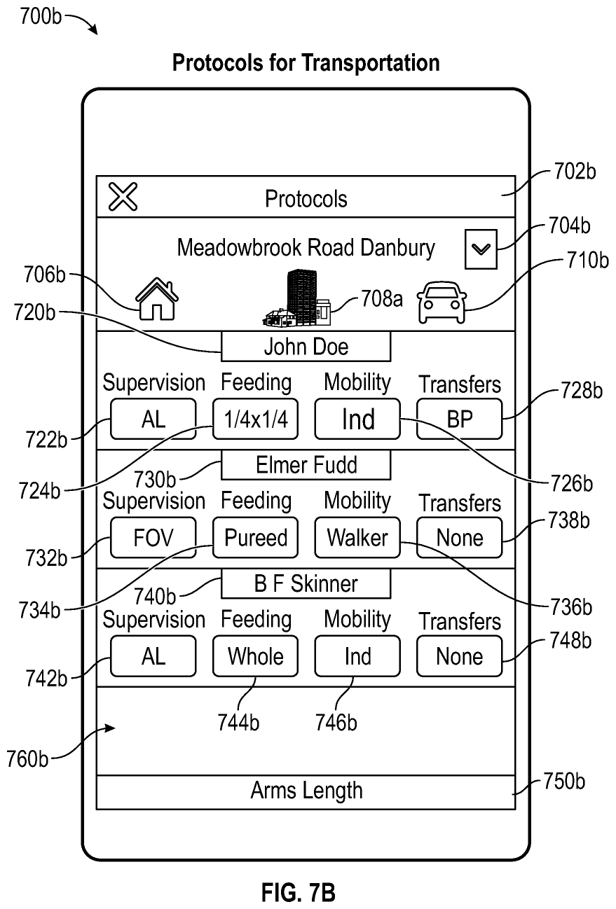

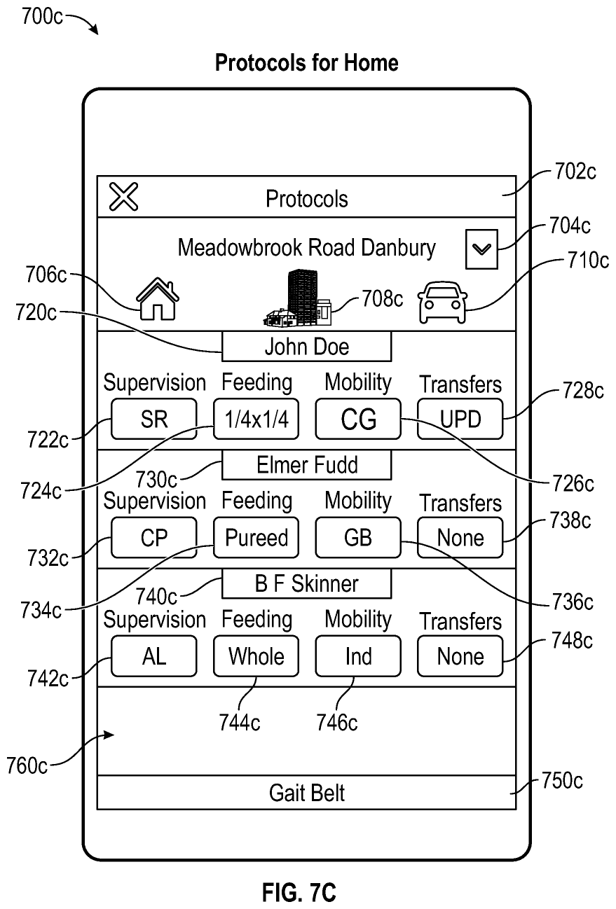

FIGS. 7A, 7B and 7C shows examples 700A, 700B, 700C of protocols according to an embodiment of the disclosure. As shown in FIGS. 7A, 7B and 7C devices showing data related to: protocols for home 702a; protocols for community 702b; and protocols for transportation 702c, respectively, are shown. The devices shown as 702a, 702b and 702c could be the same device that displays the desired data. Each device 702 generally has a display 760. Indeed, a user device with a suitable display and output functionality could be used to display any number of patient data.

FIG. 7A shows that device 702a has display 760a.

Data display 760a of device 702a shows community data includes a function icon 704a to change display data between various locations. Icon 706a indicates display of home protocol. Icon 708a indicates a community protocol and 710a represents a transportation protocol. A first patient profile for a first patient “John Doe” is shown as 720a, a second patient profile for a second patient “Elmer Fudd” is shown as 730a. A third patient profile for a third patient “B. F. Skinner” is shown as 740a. Any number of patient profiles can be displayed on the device, and accessed by scrolling through the profiles until a desired patient is viewed.

Associated with each patient name 720a, 730a, 740a, which are displayed on the device, are selected parameters, or indicator icons. For example, associated with patient John Doe 720a are indicators and data related to, for example, supervision levels 722a, feeding 724a, mobility 726a and transfers 728a.

Data indicator 722a for supervision level shows that “SR” is the supervision level, which is an abbreviation of the protocol level name. Pressing a rectangle button, which has a color associated therewith, on the screen causes the full name to show on the bottom of the screen. For example, pressing 728a “SB” causes the full name “Sliding Board” to appear in the same color (color not shown in FIGS. 7A-7C) at the bottom of the screen 750a. Pressing 750a “Sliding Board” causes detailed notes about Patient John Doe's Transfer protocol to display on the screen.

Data indicator 724a indicates the food consistency requirements for the patient John Doe.

Mobility indicator 726a provides information relating to mobility, or additional services needed to move the patient John Doe. Transfer indicator 728a indicates what type of assistance is required to safely transfer the patient John Doe.

For example, associated with patient Elmer Fudd 730a are indicators and data related to, for example, supervision level 732a, feeding 734a, mobility 736a and transfers 738a.

Data indicator 732a for supervision shows that “AL” is the supervision level for the patient John Doe.

Data indicator 734a indicates the food consistency requirements for the patient Elmer Fudd, which is indicated as “Pureed”.

Mobility indicator 736a provides information relating to mobility, or additional services needed to move the patient Elmer Fudd. Elmer Fudd is shown to require a walker. Transfer indicator 738a indicates what type of assistance the patient Elmer Fudd requires to transfer from one surface to another, such as a chair to a bed.

For example, associated with patient B. F. Skinner 740a are indicators and data related to, for example, supervision level 742a, feeding 744a, mobility 746a and transfers 748a.

Data indicator 742a for supervision shows that “AL” is the supervision level for the patient B. F. Skinner.

Data indicator 744a indicates the food consistency requirements for the patient B. F. Skinner, which is indicated as whole.

Mobility indicator 746a provides information relating to mobility, or additional services needed to move the patient B. F. Skinner. B. F. Skinner is shown to be independent. Transfer indicator 748a indicates what level of assistance the patient B. F. Skinner requires to transfer, which is indicated as none.

FIG. 7A shows that device 702a has display 760a.

Data display 760a of device 702a shows community data includes a function icon 704a to change display data between various protocols. Icon 706a indicates display of home protocol. Icon 708a indicates a community protocol and 710a represents a transportation protocol. A first patient profile for a first patient “John Doe” is shown as 720a, a second patient profile for a second patient “Elmer Fudd” is shown as 730a. A third patient profile for a third patient “B. F. Skinner” is shown as 740a. Any number of patient profiles can be displayed on the device, and accessed by scrolling through the profiles until a desired patient is viewed.

Associated with each patient name 720a, 730a, 740a, which are displayed on the device, are selected parameters, or indicator icons. For example, associated with patient John Doe 720a are indicators and data related to, for example, supervision 722a, feeding 724a, mobility 726a and transfers 728a.

Data indicator 722a for supervision shows that “SR” is the supervision level for the patient John Doe.

Data indicator 724a indicates the food consistency requirements for the patient John Doe.

Mobility indicator 726a provides information relating to mobility, or additional services needed to move the patient John Doe. Transfer indicator 728a what level of assistance the patient John Doe requires to transfer.

For example, associated with patient Elmer Fudd 730a are indicators and data related to, for example, supervision 732a, feeding 734a, mobility 736a and transfers 738a.

Data indicator 732a for supervision shows that “AL” is the supervision level for the patient John Doe.

Data indicator 734a indicates the food consistency requirements for the patient Elmer Fudd, which is indicated as pureed.

Mobility indicator 736a provides information relating to mobility, or additional services needed to move the patient Elmer Fudd. Elmer Fudd is shown to be a walker. Transfer indicator 738a what level of assistance the patient Elmer Fudd requires to transfer.

For example, associated with patient B. F. Skinner 740a are indicators and data related to, for example, supervisor 742a, feeding 744a, mobility 746a and transfers 748a.

Data indicator 742a for supervision shows that “AL” is the supervisor, which may be initials, name, badge number, employee number or other information to correlate personnel to the patient John Doe.

Data indicator 744a indicates the food consistency requirements for the patient B. F. Skinner, which is indicated as whole.

Mobility indicator 746a provides information relating to mobility, or additional services needed to move the patient B. F. Skinner. B. F. Skinner is shown to be independent. Transfer indicator 748a what level of assistance the patient B. F. Skinner requires to transfer, which is none.

FIG. 7B shows that device 702b has display 760b.

Data display 760b of device 702b shows community data includes a function icon 704b to change display data between various protocols. Icon 706b indicates display of home protocol. Icon 708b indicates a community protocol and 710b represents a transportation protocol. A first patient profile for a first patient “John Doe” is shown as 720b, a second patient profile for a second patient “Elmer Fudd” is shown as 730b. A third patient profile for a third patient “B. F. Skinner” is shown as 740b. Any number of patient profiles can be displayed on the device, and accessed by scrolling through the profiles until a desired patient is viewed.

Associated with each patient name 720b, 730b, 740b, which are displayed on the device, are selected parameters, or indicator icons. For example, associated with patient John Doe 720b are indicators and data related to, for example, supervision 722b, feeding 724b, mobility 726b and transfers 728b.

Data indicator 722b for supervision shows that “AL” is the supervision level for the patient John Doe 720b.

Data indicator 724b indicates the food consistency requirements for the patient John Doe 720b.

Mobility indicator 726a provides information relating to mobility, or additional services needed to move the patient John Doe 720b. Transfer indicator 728b what level of assistance the patient John Doe requires to transfer.

For example, associated with patient Elmer Fudd 730b are indicators and data related to, for example, supervision 732b, feeding 734b, mobility 736b and transfers 738b.

Data indicator 732b for supervision shows that “FDV” is the supervision level for the patient Elmer Fudd 730b.

Data indicator 734b indicates the food consistency requirements for the patient Elmer Fudd, which is indicated as pureed.

Mobility indicator 736b provides information relating to mobility, or additional services needed to move the patient Elmer Fudd. Elmer Fudd is shown to be “GB”. Transfer indicator 738b what level of assistance the patient Elmer Fudd requires to transfer, which is none.

For example, associated with patient B. F. Skinner 740b are indicators and data related to, for example, supervisor 742b, feeding 744b, mobility 746b and transfers 748b.

Data indicator 742b for supervision shows that “AL” is the supervision level for the patient B. F. Skinner.

Data indicator 744b indicates the food consistency requirements for the patient B. F. Skinner, which is indicated as whole.

Mobility indicator 746b provides information relating to mobility, or additional services needed to move the patient B. F. Skinner. B. F. Skinner is shown to be independent. Transfer indicator 748b indicates what level of assistance the patient B. F. Skinner requires to transfer, which is none.

FIG. 7C shows that device 702c has display 760c.

Data display 760c of device 702c shows community data includes a function icon 704c to change display data between various protocols. Icon 706c indicates display of home protocol. Icon 708c indicates a community protocol and 710c represents a transportation protocol.

A first patient profile for a first patient “John Doe” is shown as 720c, a second patient profile for a second patient “Elmer Fudd” is shown as 730c. A third patient profile for a third patient “B. F. Skinner” is shown as 740c. Any number of patient profiles can be displayed on the device, and accessed by scrolling through the profiles until a desired patient is viewed.

Associated with each patient name 720c, 730c, 740c, which are displayed on the device, are selected parameters, or indicator icons. For example, associated with patient John Doe 720c are indicators and data related to, for example, supervisor 722c, feeding 724c, mobility 726c and transfers 728c.

Data indicator 722c for supervisor shows that “SF” is the supervision level for the patient John Doe 720c.

Data indicator 724c indicates the food consistency requirements for the patient John Doe 720c.

Mobility indicator 726c provides information relating to mobility, or additional services needed to move the patient John Doe 720c. Transfer indicator 728c indicates what level of assistance the patient John Doe requires to transfer.

For example, associated with patient Elmer Fudd 730c are indicators and data related to, for example, supervisor 732c, feeding 734c, mobility 736c and transfers 738c.

Data indicator 732c for supervision shows that “CP” is the supervision level for the patient Elmer Fudd 730c.

Data indicator 734c indicates the food consistency requirements for the patient Elmer Fudd, which is indicated as pureed.

Mobility indicator 736c provides information relating to mobility, or additional services needed to move the patient Elmer Fudd. Elmer Fudd is shown to be “GB”. Transfer indicator 738b indicates what level of assistance the patient Elmer Fudd requires to transfer.

For example, associated with patient B. F. Skinner 740c are indicators and data related to, for example, supervisor 742c, feeding 744c, mobility 746c and transfers 748b.

Data indicator 742c for supervision shows that “AL” is the supervision level for the patient B. F. Skinner.

Data indicator 744c indicates the food consistency requirements for the patient B. F. Skinner, which is indicated as whole.

Mobility indicator 746c provides information relating to mobility, or additional services needed to move the patient B. F. Skinner. B. F. Skinner is shown to be independent. Transfer indicator 748c indicates what level of assistance the patient B. F. Skinner requires to transfer, which is none.

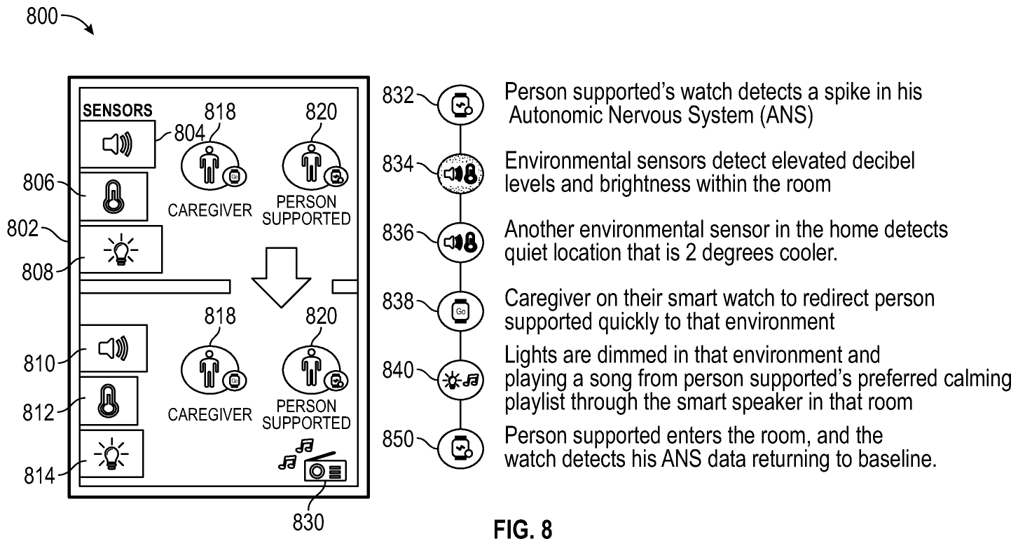

FIG. 8 shows a representation 800 of how the components act together to avert a crisis situation according to an embodiment of the present disclosure. The data representation 802 of a device, such as a smart phone, tablet, laptop, PC or other suitable device, includes sensors 804, 806, 808 that are associated with caregiver 818 and person supported 820 in a first room. Also shown as displayed data 802 are sensors 810, 812 and 814 associated caregiver 824 and a person supported 820.

Person 820 is the patient in a first room and second room. Caregiver 818 may be the same caregiver or a different caregiver.

Icon 830 shows that the environment of a patient can be controlled, such as by playing soothing music that the patient has selected on a play list, or otherwise indicated that the patient finds relaxing or calming. If there is a sensed problem, the venue is modified. One way to accomplish this is to send a signal to a device in the venue, such as an ALEXA®, SIRI® or similar device to “play music”. This signal can be generated, for example, when a patient's blood pressure exceeds a predetermined value.

As shown in FIG. 8, icon 832 shows a patient's watch detects a spike in his/her ANS. Icon 834 indicates that one or more environmental sensors detect elevated decibel levels and brightness levels within the room. Icon 836, which appears similar to icon 834, indicates that another environmental sensor, which is different than the environmental sensors 834, indicates a quiet location in proximity to the present location that is 2 degrees cooler.

Icon 838 provides an indication to a caregiver on their smart watch to redirect the person, or patient, quickly to the quiet location identified that is 2 degrees cooler. Icon 840 indicates that lights are dimmed in that environment and playing a song from the patient's, or person's, preferred calming playlist through a smart speaker in the designated room.

Icon 850 indicates the person, or patient enters the room, and the watch detects his/her ANS data returning to baseline.

Thus, as shown in FIG. 8, a first room, having sensors 804, 806 and 808 may not be a favorable venue for patient 820. Alerts associated with patient 820 indicate that patient 820 is having an adverse reaction to environment of the first room.

The patient 820 moves into a second room with sensors 810, 812 and 814. The second room may be a better environment for patient 820, as indicated by reduced sensor information.

Determinations are made for the need to intervene based on the use of an individual's own data, and their own standard deviations. This is in contrast current standard of care, which uses population parameters that indicate a threshold number that applies to all people, regardless of their individual differences, and which result in unknown quantities of false positives (interventions provided that were unnecessary and costly) and false negatives (interventions not provided that were necessary and would have prevented costly crises). Push notifications are sent within seconds of a standard deviation breach, along with instructions for the caregiver from a library of prompts.

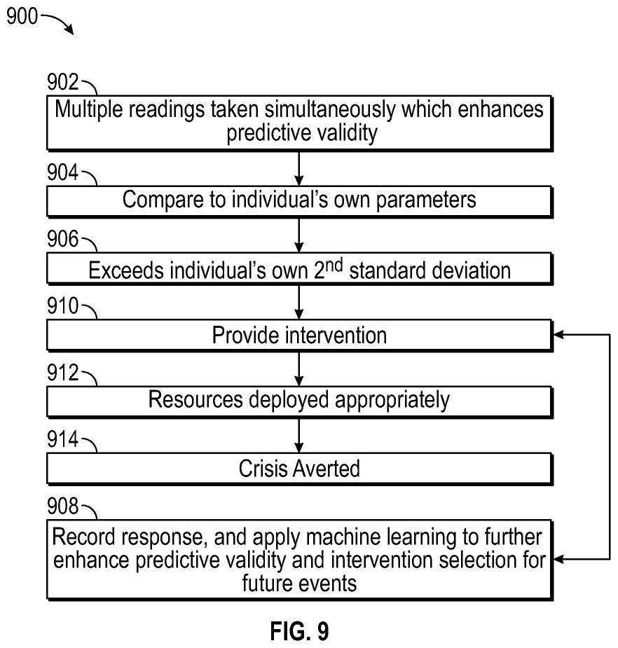

FIG. 9 shows a series of steps 900 to avert a possible crisis for a patient.

Multiple readings are taken simultaneously, or substantially simultaneously to enhance predictive validity (902). These readings include biometric data, environmental sensed data.

Biometric data includes information about the patient, such as body temperature, heart rate, blood pressure, cough symptoms and any other condition or state that can be detected by the sensor.

Environmental sensed data includes lighting, sound, temperature, motion by others, number of other people in proximity to the patient, precipitation, such as rain, lightening, thunder and/or other atmospheric and/or venue related conditions.

Acquiring data from sensors, such as biometric sensors and environmental sensors provides an indication of a present condition of a patient. The present condition is compared to a patient's individual parameters (904). The individual parameters are data points, data and/or information associated with the patient that have been acquired or obtained previously. The time of obtaining these individual specific parameters may be any time frame that the data for the patient was available. This includes years of sensed data as well as a complete medical history, such as treatments, prescriptions, vaccinations, prior episodes of stroke, seizure, heart attack and similar medical history information.

A determination is made whether the recently acquired data exceeds the individual's own second standard deviation (906). This determination is based on a prior calculation of the individual's prior medical status, or anxiety level. The computation of the standard deviations, such as first, second, third, fourth, etc. is associated with the individual patient and accessed for the determination of whether the sensed parameters exceed a first standard deviation, a second standard deviation, a third standard deviation, etc.

In the event that the acquired data parameters exceed a second standard deviation for the patient, an intervention is provided (910). The intervention is addressed and documented by recording response and applying machine learning to further enhance predictive validity and intervention selection for future events (908).

Resources are deployed appropriately (912) and the crisis is averted (914).

Embodiments of the present disclosure relate to mitigating Anxiety Disorders, stress and other underlying or apparent symptoms of emotional difficulty or instability, Post-Traumatic Stress Disorder (PTSD), Disruptive Behavior Disorders, Bipolar Disorder, and Developmental Disabilities, among other conditions. The sensors as described herein enable patient's to be monitored prior to a crisis situation. The ability to suggest a patient or person (e.g. FIG. 8, element 820) move from one area, venue or room to another area, venue or room based on physical symptoms that are related to the specific patient or person, mitigates a possible crisis situation. The physical symptoms may include elevated heart rate, elevated body temperature, lethargy, cough and/or other detectable symptoms.

Alternate embodiments, as described herein, relate to connectivity of IoT (Internet of Things) sensor devices including environmental sensors, wearable biometric sensors, and comprehensive electronic health record data via a secure and encrypted data connection stored in a data warehouse (shown in FIG. 1).

US Pre-Grant Publication 2020005290 entitled, “Intelligent Teleconference Operations in an Internet of things IoT Computing Environment”, which is hereby incorporated by reference in its entirety herein describes that IoT computing devices may be embedded in objects, especially appliances, and connected through a network. An IoT network may include one or more IoT devices or “smart devices”, which are physical objects such as appliances with computing devices embedded therein. Examples of network-enabled appliances or devices may include computers, smartphones, laptops, wearable devices, sensor devices, voice-activated devices, face-activated devices, digital assistants, home appliances, audio systems, televisions, security cameras, security sensors, among countless other examples. Such IoT computing systems may be employed in a variety of settings.

Indeed, US Pre-Grant Publication 2020005290 entitled, “Intelligent Teleconference Operations in an Internet of things IoT Computing Environment”, which is hereby incorporated by reference in its entirety herein describes that immediate, real-time communication enables various user equipment (“UE”) such as, for example, a computing device/wireless communication device (e.g., the IoT device) to share communications, such as conference calls (e.g., audio and/or video conference calls), messages, chat messages, emails, speeches, social media posts, and other content to a variety of other users.

Artificial intelligence (A.I.) may be used to facilitate responses to symptoms. U.S. Pat. No. 8,126,832, entitled, “Artificial Intelligence System” is hereby incorporated by reference in its entirety herein.

In addition to A.I., machine learning may also be used to facilitate execution of the data gathering, processing and displaying data. Machine learning is disclosed in U.S. Pat. No. 10,685,188, entitled “Systems and Methods for Training Machine Learning Models for Language Clusters”, which is hereby incorporated by reference in its entirety herein.

As described herein, one embodiment is directed to a method, comprising: accessing biometric sensed data associated with biometric data of a person; accessing environmental sensed data associated with an environment of the person; accessing a priori data associated with the person; determining a standard deviation model for each person based, at least in part, on the a priori data associated with the person; determining, based at least in part on the biometric sensed data, the environmental sensed data and the standard deviation model, a status of the person; displaying a representation of the status of the person; applying an intervention treatment to the person based at least in part on the status of the person; determining a level of validity of the intervention treatment, based at least in part on a second status of the person, second biometric sensed data and second environmental sensed data; generating response data from the person, the response data based, at least in part, on the level of validity of the intervention treatment; updating the a priori data based at least in part on the response data; generating an updated standard deviation model based at least in part on the updated a priori data; and utilizing the updated standard deviation model during a subsequent determination of the status of the person.

Another embodiment is directed to the method described above, further comprising: connecting one or more biometric sensors to a processing device via a wireless connection; and transmitting the biometric sensed data from the person to the processing device via the wireless connection.

Another embodiment is directed to the method described above, further comprising: creating a plurality of electronic signals with magnitudes corresponding to different stress levels, based at least in part on the biometric sensed data and the environmental sensed data; and transmitting the electronic signals to a caregiver device.

Another embodiment is directed to the method described above, further comprising transmitting the status of the person to a caregiver device.

Another embodiment is directed to the method described above, further comprising measuring: the occurrence of problematic and pro-social target behaviors; the frequency; intensity; duration; location; and latency of the occurrence of the problematic and the pro-social behaviors; and storing the measured occurrences in a data warehouse.

Another embodiment is directed to the method described above, further comprising accessing a library of pre-programmed prompts that are related to types of behavior that are provided via an electronic interface to the person.

Another embodiment is directed to the method described above, further comprising: accessing an electronic interface that allows the person supported or a caregiver to acknowledge and record execution of one or more of the prompts.

Another embodiment is directed to the method described above, further comprising: an acknowledgement signal transmitted from the person or the caregiver that indicates presence or absence of behavior after a prompt has been executed.

Another embodiment is directed to a specific behavior recording app that measures the occurrence of problematic and pro-social target behaviors, the frequency, intensity, duration, location, and latency of these behaviors stored in a data warehouse (shown FIG. 2).

Yet another embodiment is directed to an administrative portal for the behavior recording app that allows for target behaviors, operational definitions of said target behaviors, base lining periods for the target behaviors, during which mean and standard deviations are computed.

Yet another embodiment is directed to an electronic interface that allows for entry of essential data for the behavior recording app.

Yet another embodiment is directed to data within the data warehouse and the proprietary behavior recording app generates a behavior profile for the patient supported indicating the potential and probability of specific high risk behaviors.

Yet another embodiment is directed to machine learning that computes predictive modeling per individual identifying when problematic behaviors are likely to occur, using independent variables.

Yet another embodiment is directed to a library of pre-programmed prompts that are related to functions of behavior that will be administered via an electronic interface to the person supported, as shown in FIG. 3.

Yet another embodiment is directed to a library of pre-programmed prompts that are related to functions of behavior that will be administered via an electronic interface to a caregiver if the person supported is unable to understand and execute prompts independently, as shown in FIG. 3.

Yet another embodiment is directed to providing coding of prompts by functions of the behavior that allows the machine learning to iterate more quickly.

Yet another embodiment is directed to an electronic interface that allows for the person supported or caregiver to acknowledge and record execution of the prompt.

Yet another embodiment is directed to an electronic interface that allows for the person supported or caregiver to acknowledge and record presence or absence of behavior after a prompt has been executed.

Yet another embodiment is directed to machine learning that identifies which prompts result in behavior reduction for the individual as measured and under which contexts as measured by items.

Yet another embodiment is directed to machine learning to identify similarities between end users, thus accelerating the selection of the correct intervention for future for future similar users.

Yet another embodiment is directed to machine learning that permits people to become independent through prompting (as shown in FIG. 3).

Yet another embodiment is directed to machine learning that calculates the mean and standard deviation for autonomic nervous system data collected through biometric sensors.

Yet another embodiment is directed to a behavior data collection system that calculates the person's own mean, and standard deviation for their behaviors (as shown in FIG. 2).

Yet another embodiment is directed to the behavior data collection system that allows for differentiation between behaviors that are desired to increase, and behaviors that are desired to decrease.

Yet another embodiment is directed to the behavior data collection system that sends push notifications to the user when their behavior exceeds 3 standard deviations in any day, 2 standard deviations in 2 of 3 consecutive days, or when there are 5 consecutive increasing (or decreasing for behaviors desired to increase) data points (as shown in FIG. 4).

Yet another embodiment is directed to an autonomic nervous system measurement from devices that provides visualizations in real-time on a phone, tablet, computer, or other electronic device, plotting the mean, standard deviation (as shown FIG. 5).

Yet another embodiment is directed to a system that includes push notifications that are sent to the person supported and caregiver via electronic interface when a person's Autonomic Nervous System data exceeds the third standard deviation (as shown in FIG. 6).

Yet another embodiment is directed to the system, as described above that utilizes visualizations that have marks to interpret the data (i.e., upper and lower control limits) to understand when a person's autonomic nervous system is in a hyper and hypo aroused state relative to their own baseline (as shown in FIG. 5).

Yet another embodiment is directed to an embodiment in which a person supported or a caregiver may utilize the visualizations in order to conduct exposure therapy trials with objective data.

Yet another embodiment is directed to an embodiment in which notifications can be sent to the person supported by the technology for any sensor data point which exceeds the upper or lower control limit (i.e., 3 standard deviations).

Yet another embodiment is directed to an embodiment in which notifications can be sent to caregivers for any sensor data point, which exceeds the upper or lower control limit.

Yet another embodiment is directed to an embodiment in which machine learning identifies when environmental conditions increase the probability of problematic target behaviors and modifies these environmental conditions (for example, but not limited to, reducing the ambient temperature or lighting).

Yet another embodiment is directed to a psychological profile that can be entered into the data warehouse for each caregiver.

Yet another embodiment is directed to an embodiment in which machine learning identifies which type of psychological profile of caregiver is optimal for each person supported by the technology.

Yet another embodiment is directed to a Protocol app that provides visualizations that summarize the ways in which caregivers are to provide supports (i.e., food preparation, supervision levels, assistance with mobility) to people, as shown in FIGS. 7A-7C.

Yet another embodiment is directed to an electronic interface that summarizes each protocol for caregiver staff so that they can reference this quickly in real-time as needed, as shown in FIGS. 7A-7C.

Yet another embodiment is directed to protocols that are structured in a ranked order, using, for example, a rainbow color scheme (color not shown in figures) such that one color, such as the color “red”, may be used to equate to the highest level of acuity/risk, and a second color, such as the color “purple”, may be used to equate to the lowest. The protocols have initials to abbreviate the names, and clicking them will provide the full details on the bottom of the screen, as shown in FIGS. 7A-7C.

Yet another embodiment is directed to an administrative portal that allows protocols to be modified as needed, with 6 structured levels of intervention for each type of support provided.

Some of the illustrative embodiments of the present disclosure may be advantageous in solving the problems herein described and other problems not discussed which are discoverable by a skilled artisan. While the above description contains much specificity, these should not be construed as limitations on the scope of any embodiment, but as exemplifications of the presented embodiments thereof. Many other variations are possible within the teachings of the various embodiments. While the disclosure has been described with reference to exemplary embodiments, it will be understood by those skilled in the art that various changes may be made and equivalents may be substituted for elements thereof without departing from the scope. In addition, many modifications may be made to adapt a particular situation or material to the teachings without departing from the essential scope thereof.

Therefore, it is intended that the disclosure not be limited to the particular embodiment disclosed as the best or only mode contemplated for carrying out this invention, but that the disclosure will include all embodiments falling within the scope of the appended claims. Also, in the drawings and the description, there have been disclosed exemplary embodiments and, although specific terms may have been employed, they are unless otherwise stated used in a generic and descriptive sense only and not for purposes of limitation, the scope of the disclosure therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item. Thus, the scope of the disclosure should be determined by the appended claims and their legal equivalents, and not by the examples given.

1. A method, comprising:
accessing biometric sensed data associated with biometric data of a person;
accessing environmental sensed data associated with an environment of the person;
accessing a priori data associated with the person;
determining a standard deviation model for each person based, at least in part, on the a priori data associated with the person;
determining, based at least in part on the biometric sensed data, the environmental sensed data and the standard deviation model, a status of the person;
displaying a representation of the status of the person;
applying an intervention treatment to the person based at least in part on the status of the person;
determining a level of validity of the intervention treatment, based at least in part on a second status of the person, second biometric sensed data and second environmental sensed data;
generating response data from the person, the response data based, at least in part, on the level of validity of the intervention treatment;
updating the a priori data based at least in part on the response data;
generating an updated standard deviation model based at least in part on the updated a priori data; and
utilizing the updated standard deviation model during a subsequent determination of the status of the person.

2. The method of claim 1, further comprising:
connecting one or more biometric sensors to a processing device via a wireless connection; and
transmitting the biometric sensed data from the person to the processing device via the wireless connection.

3. The method of claim 1, further comprising:
creating a plurality of electronic signals with magnitudes corresponding to different stress levels, based at least in part on the biometric sensed data and the environmental sensed data; and
transmitting the electronic signals to a caregiver device.

4. The method of claim 1, further comprising transmitting the status of the person to a caregiver device.

5. The method of claim 1, further comprising measuring: the occurrence of problematic and pro-social target behaviors; the frequency; intensity; duration; location; and latency of the occurrence of the problematic and the pro-social behaviors; and
storing the measured occurrences in a data warehouse.

6. The method of claim 1, further comprising accessing a library of pre-programmed prompts that are related to types of behavior that are provided via an electronic interface to the person.

7. The method of claim 6, further comprising accessing an electronic interface that allows the person supported or a caregiver to acknowledge and record execution of one or more of the prompts.

8. The method of claim 7, further comprising transmitting an acknowledgement signal from the person or the caregiver that indicates presence or absence of behavior after a prompt has been executed.
